(19) United States
(12) Patent Application Publication
(10) Pub. No.: US 2008/0207989 A1
(43) Pub. Date: Aug. 28, 2008

(54) System For Positioning Support Mesh in a Patient

(75) Inventors: Richard C. Kaleta, Plymouth, MN (US); Robert E. Lund, St. Michael, MN (US); Jose' W. Jimenez, Apple Valley, MN (US); Matthew J. Olson, Crystal, MN (US); John F. Otte, St. Anthony, MN (US); Jason Westrum Ogdahl, Minneapolis, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/064,173

(22) PCT Filed: Aug. 29, 2006
(86) PCT No.: PCT/US2006/033508

§ 371 Date: Feb. 19, 2008

Related U.S. Application Data
(60) Provisional application No. 60/712,364, filed on Aug. 29, 2005.

(51) Int. Cl. A61F 2/00

(52) U.S. Cl. 600/37; 606/151

(57) ABSTRACT

A surgical system for positioning organs in a patient. The surgical system includes a mesh retrieval device and a mesh support structure. The mesh support structure is suitable for implanting in the patient to position the organ at a desired location in the patient. The mesh retrieval device is engageable with the mesh support structure. The system includes specialized needles and cannulas for delivery of the mesh retrieval device to the mesh support structure and the subsequent positioning of the mesh support structure, which results in the positioning of the organ. Methods of positioning organs in a patient using such devices are also disclosed.

### RELATED APPLICATIONS

This application is a non-provisional application of U.S. Provisional Application No. 60/712,364, filed Aug. 29, 2005, which is incorporated by reference.

### BACKGROUND OF THE INVENTION

1. Field of the Invention

The invention relates generally to treatments for positioning organs in a patient. More particular, the invention relates to devices and surgical techniques for use in treating female incontinence and prolapsed tissues.

2. Description of the Related Art

Over 13 million American men and women of all ages suffer from urinary incontinence. The social implications for an incontinent patient include loss of self-esteem, embarrassment, restriction of social and sexual activities, isolation, depression and, in some instances, dependence on caregivers. Incontinence is the most common reason for institutionalization of the elderly.

Incontinence may occur when the muscles of the urinary system malfunction or are weakened. Other factors, such as trauma to the urethral area, neurological injury, hormonal imbalance or medication side-effects, may also cause or contribute to incontinence. There are five basic types of incontinence: stress incontinence, urge incontinence, mixed incontinence, overflow incontinence, and functional incontinence. Stress urinary incontinence (SUI) is the involuntary loss of urine that occurs due to sudden increases in intra-abdominal pressure resulting from activities such as coughing, sneezing, lifting, straining, exercise and, in severe cases, even simply changing body position. Urge incontinence, also termed “hyperactive bladder” “frequency/urgency syndrome” or “irritable bladder,” occurs when an individual experiences the immediate need to urinate and loses bladder control before reaching the toilet. Mixed incontinence is the most common form of urinary incontinence. Inappropriate bladder contractions and weakened sphincter muscles usually cause this type of incontinence. Mixed incontinence is a combination of the symptoms for both stress and urge incontinence. Overflow incontinence is a constant dripping or leakage of urine caused by an overfilled bladder. Functional incontinence results when a person has difficulty moving from one place to another. It is generally caused by factors outside the lower urinary tract, such as deficits in physical function and/or cognitive function.

Stress urinary incontinence is generally thought to be related to hypermotility of the bladder neck or an intrinsic urethral sphincter defect.

A variety of treatment options are currently available to treat incontinence. Some of these treatment options include external devices, behavioral therapy (such as biofeedback, electrical stimulation, or Kegal exercises), injectable materials, prosthetic devices and/or surgery. Depending on age, medical condition, and personal preference, surgical procedures can be used to completely restore continence.

Conservative management of stress urinary incontinence can include lifestyle changes, such as weight loss, smoking cessation, and modification of intake of diuretic fluids such as coffee and alcohol. With regard to surgical treatments, the purported “gold standard” is the Burch Colposuspension, in which the bladder neck is suspended. Mid-urethral slings have been similarly effective. One type of procedure, found to be an especially successful treatment option for SUI in both men and women, is a sling procedure.

A sling procedure is a surgical method involving the placement of a sling to stabilize or support the bladder neck or urethra. There are a variety of different sling procedures. Slings used for pubovaginal procedures differ in the type of material and anchoring methods. In some cases, the sling is placed under the bladder neck and secured via suspension sutures to a point of attachment (e.g. bone) through an abdominal and/or vaginal incision. Examples of sling procedures are disclosed in U.S. Pat. Nos. 5,112,344; 5,611,515; 5,842,478; 5,860,425; 5,899,909; 6,039,686, 6,042,534, 6,110,101, and 6,652,450, all of which are herein incorporated by reference.

Urinary incontinence is often associated with prolapse of pelvic organs in females. Female pelvic organ prolapse has long plagued women. It is estimated by the U.S. National Center for Health Statistics that 247,000 operations for pelvic organ prolapse were performed in 1998. With the increasing age of the U.S. population, these problems will likely assume additional importance.

Prolapse may also be called uterine prolapse, genital prolapse, uterovaginal prolapse, pelvic relaxation, pelvic floor dysfunction, urogenital prolapse or vaginal wall prolapse. Pelvic organ prolapse occurs when the pelvic floor muscles become weak or damaged and can no longer support the pelvic organs. The womb (uterus) is the only organ that actually falls into the vagina. When the bladder and bowel slip out of place, they push up against the walls of the vagina. While prolapse is not considered a life threatening condition it may cause a great deal of discomfort and distress. The types of pelvic organ prolapse that can occur include prolapse of the anterior vaginal wall, cystocele (bladder prolapse), cystourethrocele, urethrocele (prolapse of the urethra), prolapse of the posterior vaginal wall, enterocele (prolapse of the small bowel), rectocele (prolapse of the rectum or large bowel), uterine prolapse, and vaginal vault prolapse (apical or top). Often, multiple organs are involved in a prolapse.

The etiology of pelvic organ prolapse is not clear, but it is related to weakening or stretching of the pelvic muscle and ligaments that support the organs in their normal position. Risk factors include pregnancy, ageing, obesity, large fibroids or tumours, chronic coughing or straining, heavy lifting, certain genetic conditions (such as collagen deficiency due to Marfan or Ehlers-Danlos syndrome), previous pelvic surgery, spinal cord conditions and injury, and ethnicity. Depending on the particular type of prolapse, pelvic organ prolapse can lead to discomfort, urinary incontinence, fecal incontinence, incomplete emptying of the bladder, tenesmus, or constipation.

Many techniques have been tried to correct or ameliorate the prolapse and its symptoms, with varying degrees of success. Nonsurgical treatment of prolapse involves measures to improve the factors associated with prolapse, including treating chronic cough, obesity, and constipation. Other nonsurgical treatments may include pelvic muscles exercises or supplementation with estrogen. These therapies may alleviate symptoms and prevent worsening, but the actual hernia will remain. Vaginal pessaries are the primary type of nonsurgical treatment, but there can be complications due to vaginal wall ulceration.

There are a variety of known surgical techniques for the treatment of pelvic organ prolapses. Both abdominal and vaginal approaches are utilized. Biological or synthetic grafts have been incorporated to augment repair. One surgical technique for the repair of anterior vaginal prolapse can be accomplished with the Perigee™ System developed by American Medical Systems located in Minnetonka, Minn. In this transobturator anterior prolapse repair procedure, an incision is made medially along the anterior vaginal wall. The bladder is dissected off the vagina up to the lateral sulcus and posterior to the vaginal vault. Two small incisions are made over the obturator membrane along the pubic remus, one superior and one inferior. Curved needles are passed from skin incisions through the obturator foramen. The superior needles pass proximal to the vaginal vault and the inferior needles pass distal to the bladder neck. The needle tips are palpated via blunt dissection as they penetrate the obturator membrane. A graft is placed across the anterior vaginal wall. Connectors pre-attached to the sheaths enclosing the self-fixating mesh appendages are attached to the left and right superior needles and retracted. This is repeated for the inferior needles. Final adjustments are made and incisions closed.

Likewise, the treatment of posterior vaginal prolapses may vary. If symptoms are minimal, nonoperative therapy such as changes in activities, treatment of constipation, and Kegel exercises might be appropriate. Again, both vaginal and abdominal approaches are used, involving sutures to reapproximate the attenuated tissue and possibly a biological or synthetic graft to augment the repair.

A surgical technique for treating a vaginal vault prolapse can be accomplished with the Apogee™ vault suspension system developed by American Medical Systems located in Minnetonka, Minn. To use the Apogee™ system, an incision is made transversely across the vaginal apex to create access to the peritoneal cavity. Two small incisions are also made in the skin of the buttocks. Needles are passed from the skin incisions in the buttocks to the vaginal incision. The needle tip is palpated distal and inferior to the ischial spine prior to passage through the coccygeus muscle. Further dissection may be desired to aid palpation of the needle passage. Connectors are attached to each needle end. Needles are retracted and mesh is positioned. The mesh is then attached to the vaginal vault and the incisions are closed.

Surgical techniques for prolapse repair often utilize surgical repair material such as a biological graft or a synthetic mesh. The biological graft can be made out of porcine dermis. A biological graft minimizes the risk of extrusions. The synthetic mesh can be made out of polypropylene. Polypropylene has desirable durability and improves usability.

As noted above, surgical treatment of urinary incontinence and prolapse genital organs can share certain features. Namely, various surgical techniques use mesh or other devices to provide needed support for some portion of the patient's anatomy, such as the bladder, bowel, or uterus. While it is possible to make a relatively large incision to provide the surgeon with direct access to the area in which the mesh support structure is being placed, this would be undesirable, as it increases the time of surgery, the pain to the patient, and the risk of other complications, such as wound dehiscence, infection, and anesthesia related problems due to prolonged surgery. As such, it is desirable to make relatively small incisions in the patient and then feed the mesh support structure to desired locations using needles or other specialized devices. However, there are several challenges associated with using needles to position the mesh support structure, the most significant relating to attaching the mesh support structure to the needle so that the mesh support structure can be readily moved inside of the patient. It is an object of the present invention to provide such specialized devices for locating mesh support structure properly.

### SUMMARY OF THE INVENTION

An embodiment of the invention is a surgical system for positioning organs in a patient. The surgical system includes a mesh retrieval device and a mesh support structure. The mesh retrieval device includes a base and at least one hook extending therefrom. The mesh support structure is suitable for implanting in the patient to position the organ at a desired location in the patient.

Another embodiment of the surgical system for positioning a mesh support structure in a patient of the present invention is a surgical system including a needle and a cannula. The needle has a handle extending therefrom. The handle facilitates insertion of the needle into the patient. The cannula has an opening formed therein that is adapted to receive at least a portion of the needle. The needle has a flange extending from an outer surface thereof.

Still another embodiment of the surgical system for a positioning support structure in a patient of the present invention is a surgical system including a mesh retrieval device, a sheath, and a needle. The mesh retrieval device has a base from which at least one hook extends. The sheath substantially encompasses the mesh retrieval device. The needle is capable of engaging at least one of the mesh retrieval device and the sheath for positioning the mesh retrieval device in the patient.

Yet another embodiment of the surgical system positioning a support structure in a patient of the present invention includes a surgical system including an inner needle portion and an outer needle portion. The inner needle portion has a tip proximate a proximal end thereof and a latch that selectively provides access to an interior of the inner needle. The outer needle portion is positionable over the inner needle portion.

Another embodiment of the surgical system for a positioning support structure in a patient of the present invention is a surgical system including a mesh retrieval device and a cannula. In this embodiment, the cannula includes a tapered edge, and portion of the cannula (in a non-limiting example, approximately half of the cannula) including the tapered edge is less rigid as compared to the remaining portions of the cannula. The more rigid aspect may be driven to the ischial spine in a surgery to place a mesh device for treatment of prolapse, with the tapered edge out the vaginal incision. The mesh implant can be conveniently attached to the mesh retrieval device and positioned.

Another embodiment of the surgical system for positioning a mesh support structure in a patient of the present invention is a surgical system including a needle, a cannula, and a sling having a leader. In this system, the needle is placed in its proper location, with a flexible canula placed into the body over the needle. The needle is subsequently removed, leaving the cannula in place. A leader formed from such materials as flexible plastic rod is attached to the sling implant to be placed. The leader is used to thread the mesh implant through the cannula to its proper location. The leader is then removed, followed by removal of the cannula after placement of the mesh implant. Alternatively, an appropriate mesh retrieval device of the present invention may be used to place the mesh into its proper location through the cannula, followed by removal of the cannula.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a perspective view of a mesh retrieval device for use in an embodiment of the invention.

FIG. 2 is a perspective view of the mesh retrieval device in a cylindrical configuration.

FIG. 3 is a perspective view of the mesh retrieval device partially extended through a hollow needle or cannula.

FIG. 4 is a perspective view showing forming a slit in the mesh retrieval device with a knife.

FIG. 5 is a perspective view showing forming a slit in the mesh retrieval device with a tear strip.

FIG. 6 is a perspective view showing an end of the mesh retrieval device in a substantially flat configuration.

FIG. 7 is a perspective view showing attachment of a mesh support structure to the mesh retrieval device.

FIG. 8 is a perspective view showing the mesh retrieval device with the mesh support structure attached thereto being pulled through the hollow needle or cannula.

FIG. 9 is a perspective view showing the mesh support structure being detached from the mesh retrieval device.

FIG. 10 is a top view of an alternate configuration for the mesh retrieval device.

FIG. 11 is a side view of the alternate configuration for the mesh retrieval device.

FIG. 12 is a side view of a first needle configuration with an associated cannula.

FIG. 13 is a side view of a second needle configuration with an associated cannula.

FIG. 14 is a side view of a third needle configuration with an associated cannula.

FIG. 15 is a side view of a cannula partially extended over a needle.

FIG. 16 is an enlarged side view illustrating the flange extending from the cannula.

FIG. 17 is a side view illustrating attachment of a sheathed mesh retrieval device attached to a first needle.

FIG. 18 is a side view illustrating attachment of a sheathed mesh retrieval device attached to a second needle.

FIG. 19 is a side view of a needle extended through the obturator foramen.

FIG. 20 is a side view illustrating removing an outer needle portion from an inner needle portion.

FIG. 21 is a side view of a latch on the inner needle portion moving to an open configuration.

FIG. 22 is a side view illustrating forming an aperture in the sacrospinous ligament with the latch.

FIG. 23 is a side view of the mesh retriever extended through the inner needle portion.

FIG. 24 is a side view of the mesh pulled through the inner needle portion.

FIG. 25 is a side view of the inner needle portion being removed from the patient.

FIG. 26 is a view of the cannula having rigid and less rigid portions engaged to a mesh retrieval device.

### DETAILED DESCRIPTION OF THE PREFERRED EMBODIMENTS

The present invention is directed to several different devices and techniques for positioning support structures in a patient, such as a patient suffering from urinary incontinence, fecal incontinence, or organ prolapse. Referring now to the drawings, wherein like reference numerals designate identical or corresponding parts throughout the several views. The following description is meant to be illustrative only, and not limiting other embodiments of this invention will be apparent to those of ordinary skill in the art in view of this description.

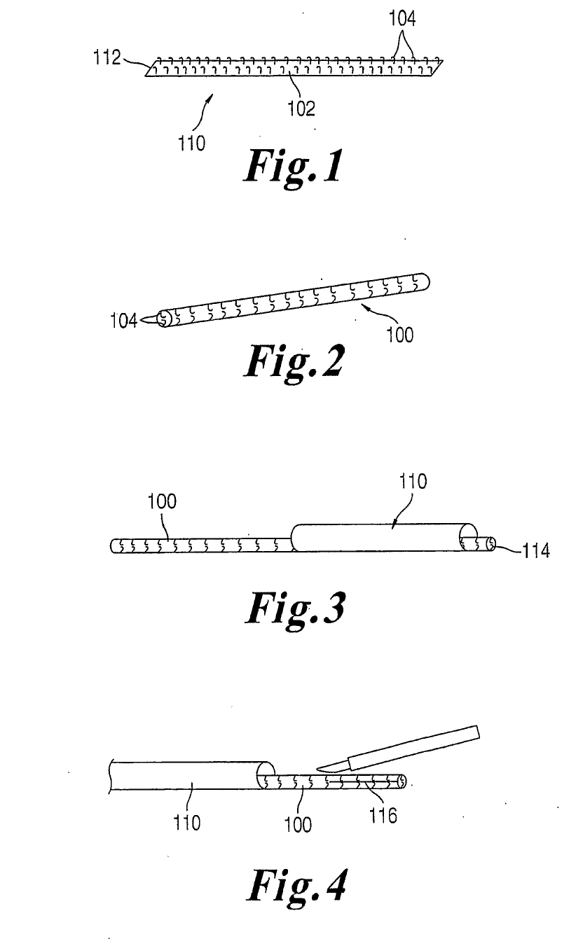

An embodiment of the invention is a surgical system for positioning organs in a patient, the surgical system including a mesh retrieval device and a mesh support structure. The mesh retrieval device includes a base and at least one hook extending therefrom. The mesh support structure is suitable for implanting in the patient to position the organ at a desired location in the patient. In this embodiment of the present invention, a mesh support structure is positioned within the body using a mesh retrieval device 100 that generally includes a base 102 from which a plurality of hooks 104 extend, as illustrated in FIG. 1. The mesh retrieval device 100 is formed from a detachable fastener such as a hook and loop fastener that is marketed under the designation VELCRO. The concepts of the present invention with alternate structures such as a ballized hook fastener that is marketed under the designation DUAL LOCK, among other alternate designs, are within the scope of the present invention. A hook portion of the hook and loop fastener is used for engaging the mesh support structure.

The base 102 and the hooks 104 may be formed from a biologically compatible material to minimize the potential of negative interactions if the base contacts the patient during use. One such suitable material is polypropylene. Each of the hooks may have a J-shaped configuration.

The mesh retrieval device 100 may be rolled into a cylindrical configuration such that the hooks 104 are inwardly directed, as illustrated in FIG. 2. The cylindrical configuration facilitates feeding the mesh retrieval device 100 into the body through a hollow needle 110, as illustrated in FIG. 3, as the exposed surface of the base 102 is substantially smooth.

A diameter of the mesh retrieval device 100 when in the cylindrical configuration may be slightly smaller than an inner diameter of the hollow needle 110 to facilitate extending the mesh retrieval device 100 through the hollow needle 110. As an alternative to using this embodiment with a hollow needle 110, a cannula may be used.

The hooks 104 may be oriented so that ends of the hooks 104 opposite the base 102 are substantially oriented towards a proximal end 112 of the mesh retrieval device 100. Orienting the hooks 104 in this manner enhances the attachment between the mesh retrieval device 100 and the mesh support structure as the mesh retrieval device 100 is pulled through the patient.

A length of the mesh retrieval device 100 is selected based upon the location within the body that the mesh support material is to be placed and is typically about 18 inches.

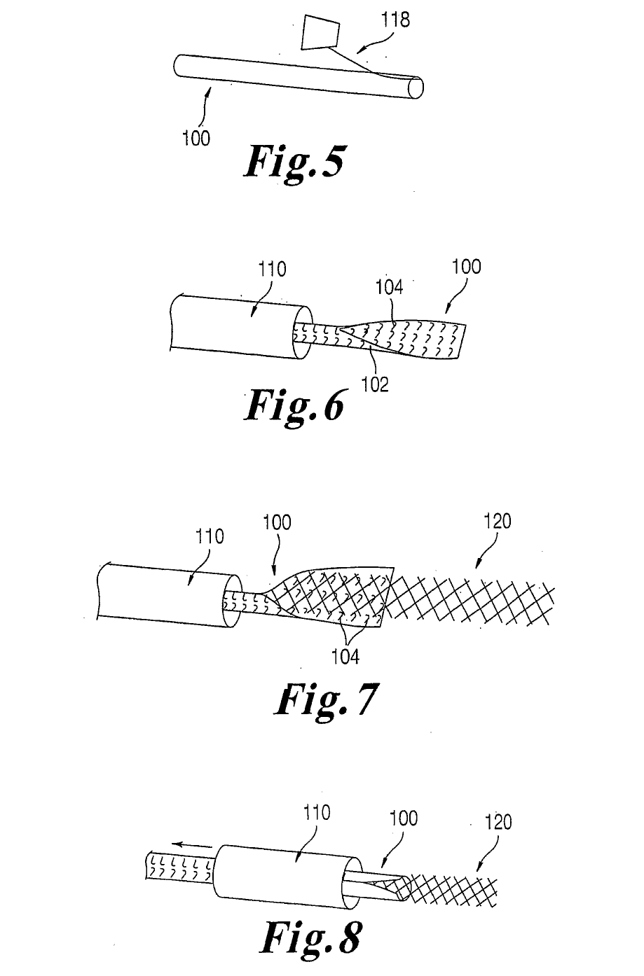

As the mesh retrieval device 100 is inserted into the patient, a distal end 114 of the mesh retrieval device 100 extends beyond a distal end 114 of the hollow needle 110, as illustrated in FIG. 4. A slit 116 is then formed in the base 102 proximate the distal end 114 of the mesh retrieval device 100 using a knife or other cutting apparatus. It is possible to form the slit 116 using alternate techniques such as a tear strip 118 or perforation, as illustrated in FIG. 5.

The slit 116 thereby allows the base 102 to be moved to a substantially flat configuration, as illustrated in FIG. 6, which exposes the hooks 104 proximate the slit 116. The mesh support structure 120 is then moved into engagement with a plurality of the hooks 104, as illustrated in FIG. 7.

The slit 116 is formed with a length that allows a sufficiently large number of hooks 104 to be exposed to provide a fastening strength that enables the mesh support structure 120 to be retained on the mesh retrieval device 100 as the mesh support structure 120 is positioned on the mesh retrieval device 100. Increasing the number of hooks 104 that engage the mesh support structure 120 results in increased attachment strength.

To enhance the ability of the mesh retrieval device 100 to move from the cylindrical configuration to a substantially flat configuration when the slit 116 is formed in the base 102, a resilient strip or wire (not shown) may be attached to the mesh retrieval device 100 proximate to the proximal end 114.

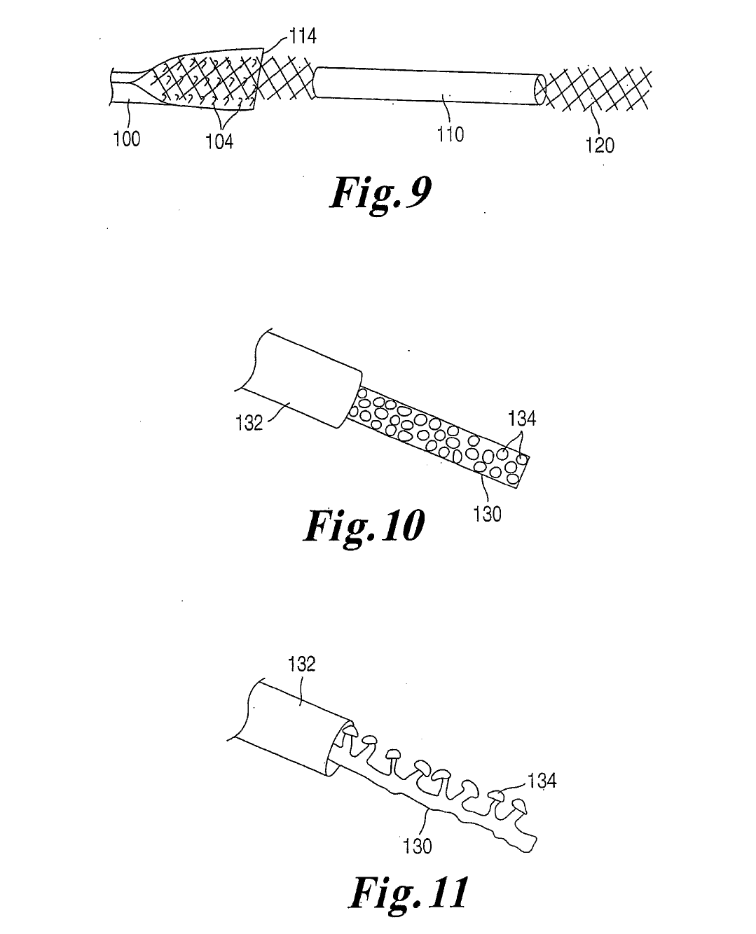

To facilitate attachment of the mesh support material 120 to the mesh retrieval device 100 and forming the slit in the mesh retrieval device 100, the distal end 114 is extended through an incision made in the vagina wall (not shown) so that the physician may grasp the distal end 114 and pull it out of the vagina. Thereafter, the mesh retrieval device 100 is urged back to the cylindrical configuration, which facilitates pulling the mesh retrieval device 100 as well as the mesh support structure 120 through the hollow needle 110, as illustrated in FIG. 8. When the distal end 114 of the mesh retrieval device 100 is pulled out of the proximal end of the hollow needle 110, the distal end 114 of the mesh retrieval device 100 is at least partially flattened so that the hooks 104 proximate the distal end 114 are exposed, as illustrated in FIG. 9. The mesh support structure 120 may then be detached from the mesh retrieval device 100 and positioned in the patient using conventionally known techniques.

This embodiment obviates the current procedure of connecting the mesh support material to a mesh placement device, such as a needle or similar device such as the devices in US Patent Publications 2005/0245787-A1 and 2005/0250977-A1 (herein incorporated by reference), within the vagina or other bodily cavity. Of course, the placement of the mesh support material through a smaller incision is allowed by the present embodiment. This embodiment thereby enhances the safety and reduces the time associated with positioning the mesh support material in the patient.

In an alternative to the embodiment illustrated in FIGS. 1-9, the mesh retrieval device 130 may be formed with a sufficiently narrow width that enables the mesh retrieval device 130 to be passed through the hollow needle 132 or cannula in a substantially flat configuration, as illustrated in FIGS. 10-11.

The hooks 134 utilized in this embodiment may have a mushroom shaped head. A mesh retrieval device 130 having these characteristics is available under the designation DUAL LOCK.

In this embodiment, the mesh retrieval device 130 may have a width of less than about 0.140 inches and may have a width of about 0.120 inches. The spacing between the rows and columns of hooks is selected based upon the pore size in the mesh support structure to facilitate forming a strong attachment between the mesh retrieval device 130 and the mesh support structure. Because of the relatively narrow shape of the mesh retrieval device 100, there are typically only one or two rows of hooks extending across the base.

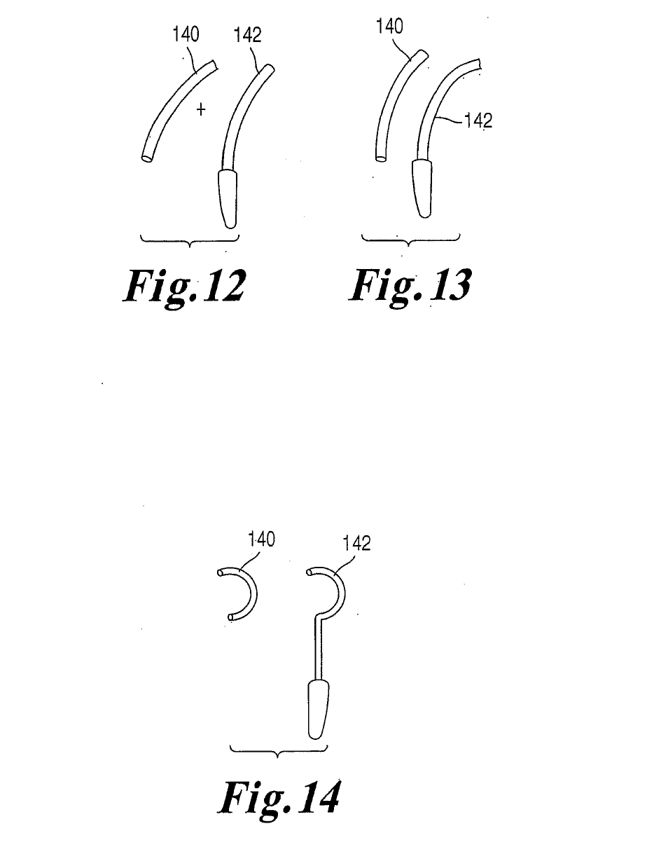

In another embodiment of the present invention relating to the surgical apparatus with which a mesh support structure is positioned in the patient, a cannula 140 is shaped to at least partially extend over the needle 142, which is used to position the cannula 140 in the body, as illustrated in FIGS. 12-14. Once the cannula 140 is inserted into the patient, the needle 142 is withdrawn from the cannula 140. The cannula 140 thereby provides a substantially cylindrical central opening that may be utilized to position the mesh support structure in the patient such as through the mesh retrieval device, which is described above with respect to FIGS. 1-9. This embodiment may be adapted for use in a variety of surgical techniques. Using the cannula 140 over the needle 142 eliminates the need to make connections between the needle 142 and mesh support structure at difficult to access locations within the patient such as in the vagina or abdomen.

Another embodiment of the surgical system for positioning a mesh support structure in a patient of the present invention is a surgical system including a needle and a cannula. The needle has a handle extending therefrom. The handle facilitates insertion of the needle into the patient. The cannula has an opening formed therein that is adapted to receive at least a portion of the needle. The needle has a flange extending from an outer surface thereof.

In this embodiment, when the cannula is used with a helical shaped needle that is commonly used to treat stress urinary incontinence or anterior vaginal prolapse, such as the needles marketed under the designation MONARC or PERIGEE, respectively, by the assignee of the present patent application, the cannula is preferably formed in a shape that is similar to a question mark. When the cannula is used with a curved needle such as is commonly used when to treat stress urinary incontinence or when performing vaginal vault prolapse repair such as are marketed under the designation SPARC or APOGEE, respectively, by the assignee of the present patent application, the cannula is preferably formed in a U-shape. Because of the flexibility of the cannula, the curvature of the cannula in the preceding applications may be provided in a two dimensional plane.

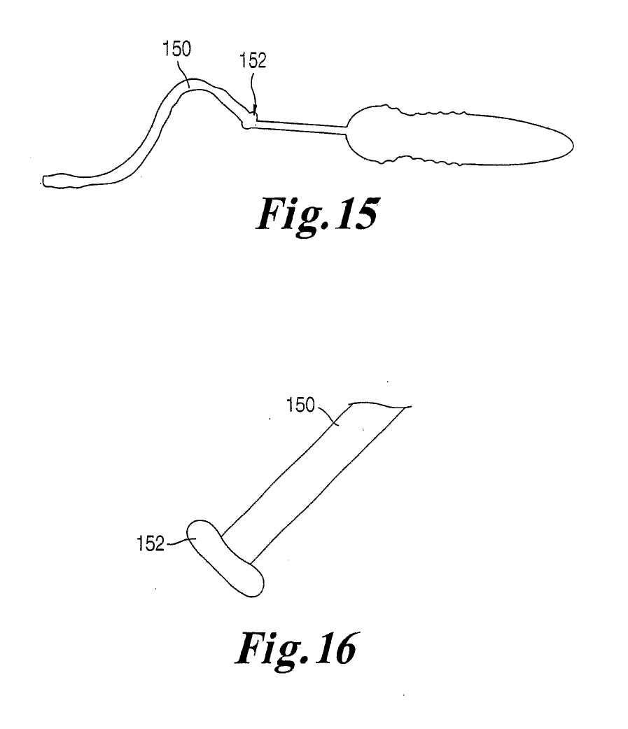

Proximate to a proximal end of the cannula, the cannula 150 in the present embodiment includes a flange 152 that extends from an outer surface thereof, as illustrated in FIGS. 15-16. The flange 152 may extend around the outer surface to form a circular collar. The flange 152 extends sufficiently far away from the outer surface of the cannula 150 to prevent the cannula from passing into the body. The flange 152 also enhances the ability to withdraw the cannula 150 from the patient. Alternate shapes for the flange 152 are within the scope of the present invention.

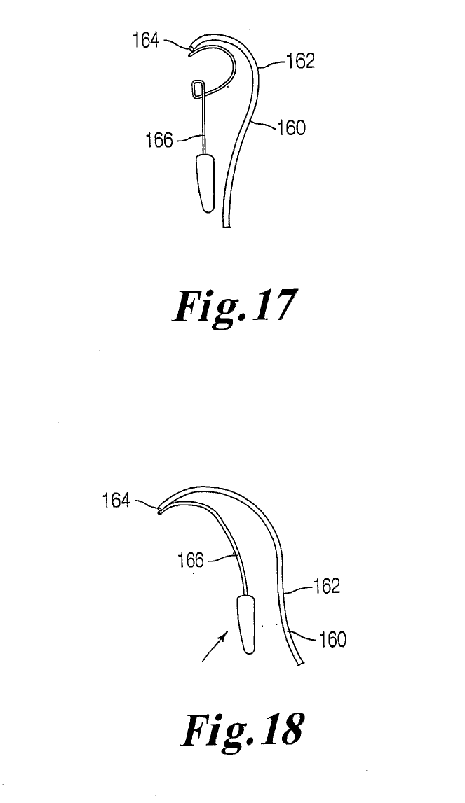

Another embodiment of the surgical system for a positioning support structure in a patient of the present invention is a surgical system including a mesh retrieval device, a sheath, and a needle. The mesh retrieval device has a base from which at least one hook extends. The sheath substantially encompasses the mesh retrieval device. The needle is capable of engaging at least one of the mesh retrieval device and the sheath for positioning the mesh retrieval device in the patient. In this embodiment, the mesh retrieval device 160 having a configuration that may be similar to the mesh retrieval device described with respect to the prior embodiments is extended through a sheath 162 such that the mesh retrieval device 160 is substantially covered by the sheath 162, as illustrated in FIGS. 17-18. Covering the mesh retrieval device 160 with the sheath 162 enables the mesh retrieval device 160 to be pulled through the patient without the hooks damaging tissue in the patient and without the use of a cannula or other similar device.

A distal end 164 of the sheath 162 may be enclosed to prevent tissue or fluid from entering the sheath 162 as the sheath 162 is pulled through the patient. The distal end 164 also has an attachment tab (not shown) that enables the sheath 162 to be attached to a needle 166 that is used to pull the sheath 162 and the mesh retrieval device 160 retained therein through the patient. The needle 166 may be shaped in a configuration to facilitate performing particular operations such as are currently available under the designations PERIGEE and APOGEE from the assignee of the present patent application.

Similar to the mesh retrieval device 160, an arm of the mesh support material used in this embodiment may be covered with a sheath to facilitate positioning the mesh support material in the patient as the mesh support material is typically fabricated from a material that encourages adhesion to tissue in the patient.

A distal end of the mesh support material arm extends from a distal end of the sheath 162 to facilitate connecting the hooks to the mesh support material. After the mesh retrieval device is attached to the mesh support material, a sheath may be placed over the region that is proximate the connection so that when the mesh support material is pulled through the patient, tissue and bodily fluid do not contact the mesh retrieval device or the mesh support material. Once the mesh support material is in a desired position, the mesh retrieval device is detached from the mesh support material and the sheath 162 is removed from the mesh support material arm.

After the needle 166 positions the covered mesh retrieval device at a desired location in the patient, the needle 166 is detached from the sheath 162 and the distal end of the sheath 162 is opened to expose the mesh retrieval device 160. To facilitate removing the distal end of the sheath 162 and attaching the mesh support material to the mesh retrieval device 160, a distal end of the mesh retrieval device 160 may be pulled out of the patient such as through the vagina. Using this technique obviates performing these tasks within the body where it may be difficult to reach and where visibility may be limited. This technique thereby enables smaller incisions to be made in the patient, which reduces the potential of complications and reduces the recovery time of the patient.

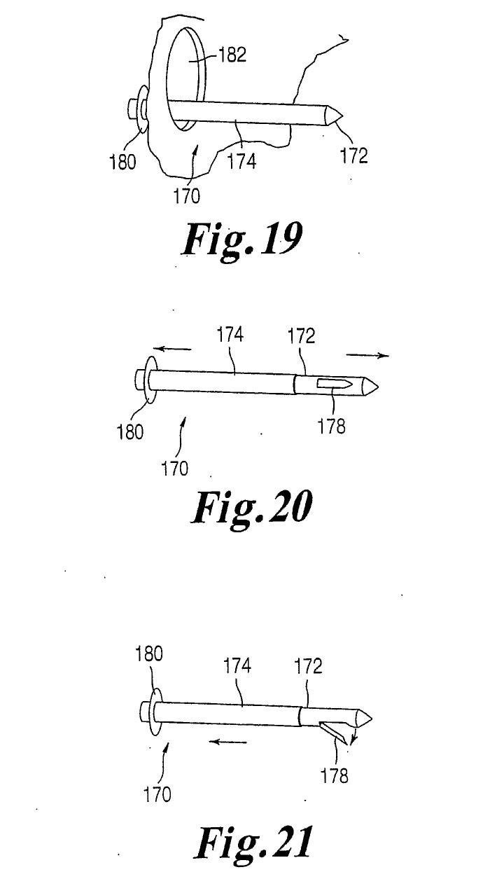

Another embodiment of the surgical system positioning a support structure in a patient of the present invention includes a surgical system including an inner needle portion and an outer needle portion. The inner needle portion has a tip proximate a proximal end thereof and a latch that selectively provides access to an interior of the inner needle. The outer needle portion is positionable over the inner needle portion. A needle 170 is used in conjunction with this embodiment of the invention, as seen in FIGS. 19-21. The needle 170 may include an inner needle portion 172 and an outer needle portion 174 that extends substantially over the inner needle portion 172, as illustrated in FIGS. 19-21.

In this embodiment, the inner needle portion 172 has a substantially hollow inner core. At a distal end of the inner needle portion 172, a tapered tip 176 is provided to facilitate feeding the needle through the tissue in the patient. Proximate the distal end, the inner needle portion 172 includes a latch 178 that is biased away from the inner needle portion 172.

When the latch 178 is in an open configuration, the latch 178 provides access to the hollow inner core for feeding a mesh retriever through the hollow inner core, as is described in more detail below. The outer needle portion 174 extends over the latch 178 and thereby maintains the latch 178 in a closed configuration that may be substantially aligned with an outer surface of the inner needle portion 172.

The outer needle portion 174 may have an inner diameter that is approximately the same as an outer diameter of the inner needle portion 172. A proximal end of the outer needle portion 174 may include a collar 180 extending radially therefrom. The collar 180 prevents the outer needle portion 174 from wholly passing inside of the patient as the needle 170 is positioned in the patient. The collar 180 also facilitates moving the outer needle portion 174 with respect to the inner needle portion 172.

The outer needle portion 174 may be formed from a variety of materials using the concepts of the present invention. One particularly suitable material for use in fabricating the outer needle portion 174 is polypropylene.

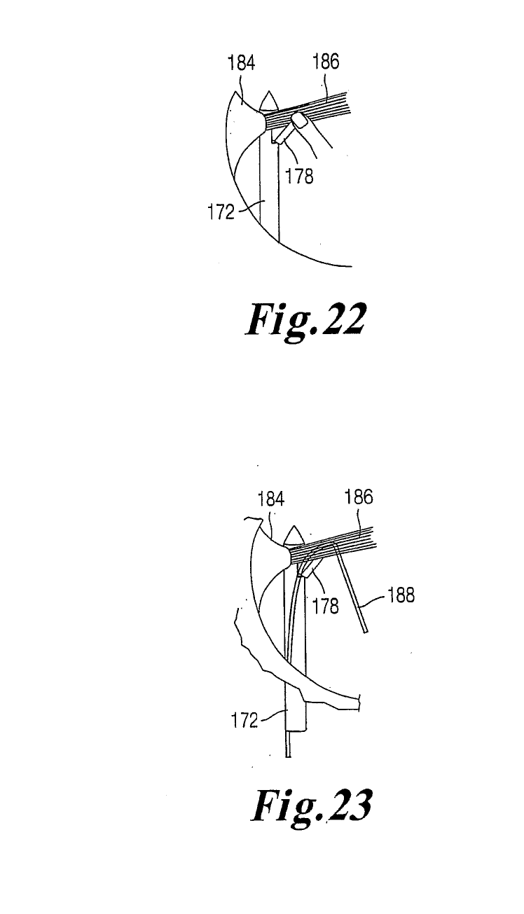

As an initial step in performing this technique, an anterior or posterior vaginal dissection is performed using conventionally known techniques. Next, the needle 170 is inserted through the obturator foramen 182 until a distal end of the needle 170 is proximate the ischial spine 184, as illustrated in FIG. 19. Next, the collar 180 is pulled away from the patient to slide the outer needle portion 174 away from the distal end of the inner needle portion 172, as illustrated in FIG. 20. As the outer needle portion 174 is moved with respect to the inner needle portion 172, the latch 178 is permitted to move to the open configuration, as illustrated in FIG. 21. The physician then palpates the latch 178 and uses the latch 178 to form an aperture in the sacrospinous ligament or ilio-coccygeus muscle 186 by extending the latch 178 through the sacrospinous ligament or ilio-coccygeus muscle 186, as illustrated in FIG. 22. To facilitate forming the aperture in the sacrospinous ligament or ilio-coccygeus muscle 186, an edge of the latch 178 may include a sharpened surface.

This technique obviates extending the needle 170 through the sacrospinous ligament or ilio-coccygeus muscle 186 by torquing the needle 170. Using the latch 170 to form the aperture thereby enhances the ability to accurately form the aperture in the sacrospinous ligament or ilio-coccygeus muscle 186 without adversely tearing tissue.

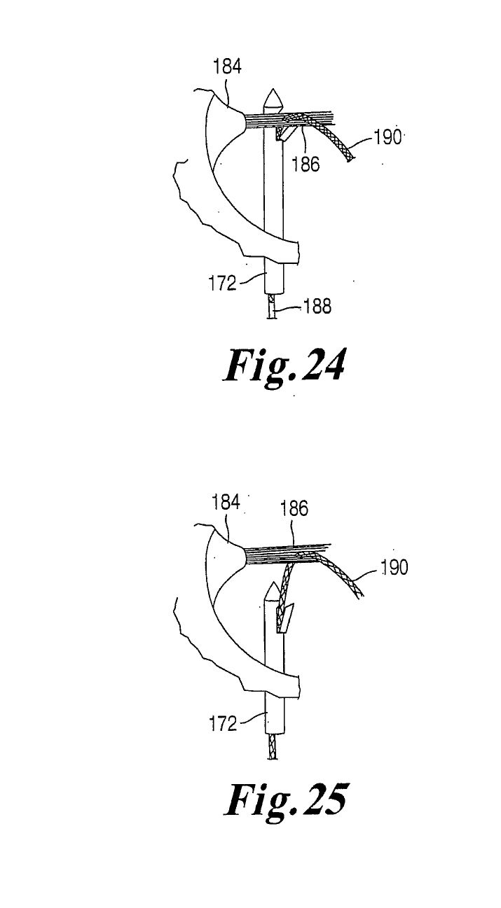

As illustrated in FIG. 23, a distal end of the mesh retrieval device 188 may be bent to facilitate attachment of the mesh retrieval device 188 to the mesh support structure. While a variety of techniques may be used to bend the mesh retrieval device 188, a particularly suitable technique for bending the mesh retrieval device 188 is heat treating. A mesh retrieval device 188 is then fed through the hollow inner core until the mesh retriever 188 extends out of the opening that was covered by the latch 178. The mesh retriever 188 then passes through the aperture in the sacrospinous ligament or ilio-coccygeus muscle 186. Next, the mesh retrieval device 188 is extended through the anterior or posterior vaginal dissection. The bent end of the mesh retrieval device 188 may be bent back to its original substantially straight configuration to pass the mesh retrieval device 188 through the anterior or posterior vaginal dissection. To facilitate connecting the mesh to the mesh retrieval device 188, the mesh retrieval device 188 is pulled to a location outside of the vagina to facilitate connecting the mesh support structure to the mesh retrieval device 188. Once the mesh is attached to the mesh retriever, the mesh retriever is pulled through the patient to position the mesh at a desired location in the patient, as illustrated in FIG. 24. The inner needle portion is removed from the patient, as illustrated in FIG. 25. Thereafter, the mesh retriever is detached from the mesh and the mesh is affixed inside of the patient using conventionally known techniques.

This technique enables the physician to connect the mesh to the mesh transport outside of the vagina, which thereby enables the physician to have more space for making the location and to visually confirm the connection is correct.

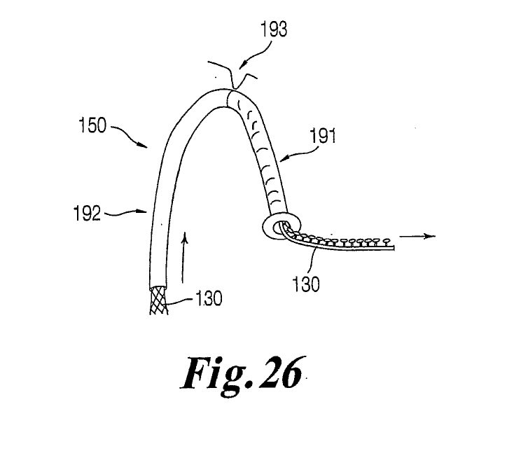

Another embodiment of the surgical system for a positioning support structure in a patient of the present invention is a surgical system including a mesh retrieval device and a cannula. In this embodiment, the cannula includes a tapered edge, and portion of the cannula (in a non-limiting example, approximately half of the cannula) including the tapered edge is less rigid as compared to the remaining portions of the cannula. The more rigid aspect may be driven to the ischial spine in a surgery to place a mesh device for treatment of prolapse incontinence, with the tapered edge out the vaginal incision. The mesh implant can be conveniently attached to the mesh retrieval device and positioned. This embodiment is illustrated in FIG. 26. FIG. 26 shows the cannula 150 having a first end 191 that is substantially rigid and a second end 192 distal to the first end. The rigid first end 191 is driven to near the ischial spine 193, and the second end 192 is coming out of a vaginal incision or opening (not shown). A mesh retrieval device 130 is engaged with the cannula for placement of the mesh implant.

The various embodiments of the present invention are also applicable to the treatment of male incontinence and pelvic floor muscle support in men via a perineal incision. The various embodiments can also be introduced via a single transvaginal or perineal incision (in males).

It is contemplated that features disclosed in this application, as well as those described in the above applications and publications incorporated by reference, can be mixed and matched to suit particular circumstances. In addition, numerous modifications and variations of the present invention are possible in light of the above teachings. It is therefore to be understood that within the scope of the appended claims, the invention may be practiced otherwise than as specifically described herein.

1. A surgical system for positioning organs in a patient, the surgical system comprising:
a mesh retrieval device comprising a base and at least one hook extending therefrom; and
a mesh support structure adapted to be implanted in the patient to position the organ at a desired location in the patient, wherein
the mesh retrieval device is adapted to engage the mesh support structure.

2. The surgical system of claim 1, wherein the mesh retrieval device is fabricated from a hook portion of a hook and loop fastener or from a ballized fastening device.

3. The surgical system of claim 1, wherein the mesh retrieval device is oriented in a substantially cylindrical configuration.

4. The surgical system of claim 3, wherein the hooks extend towards an interior of the mesh retrieval device.

5. The surgical system of claim 1, wherein the mesh retrieval device has a tear strip or perforation that facilitates forming a slit in the base.

6. The surgical system of claim 1, and further comprising a cannula through which the mesh retrieval device may be inserted.

7. The surgical system of claim 6, wherein the base has a substantially flat configuration when the mesh retrieval device is inserted through the cannula.

8. The surgical system of claim 6, wherein the cannula comprises
a first portion substantially rigid, and
a second portion having a tapered edge, said portion being distal to the first portion and less rigid than said first portion.

9. A surgical system for positioning a mesh support structure in a patient, the surgical system comprising:
a needle including a handle extending therefrom, wherein the handle facilitates insertion of the needle into the patient; and
a cannula including an opening formed therein that is adapted to receive at least a portion of the needle.

10. The surgical system of claim 9, wherein the needle has a flange extending from an outer surface thereof.

11. The surgical system of claim 10, wherein the flange is positioned proximate to a proximal end of the cannula.

12. The surgical system of claim 9, further comprising:
a leader for engagement of said mesh support structure to facilitate placement through said cannula.

13. A surgical system for a positioning support structure in a patient, the surgical system comprising:
a mesh retrieval device including a base from which at least one hook extends;
a sheath that substantially encompasses the mesh retrieval device; and
a needle that is adapted to engage at least one of the mesh retrieval device and the sheath for positioning the mesh retrieval device in the patient.

14. The surgical system of claim 13, wherein the mesh retrieval device is fabricated from a hook portion of a hook and loop fastener or from a ballized fastening device.

15. The surgical system of claim 13, wherein the mesh retrieval device is oriented in a substantially cylindrical configuration.

16. The surgical system of claim 13, wherein said at least one hook extends toward an interior of the mesh retrieval device.

17-19. (canceled)
